(19) United States
(12) Patent Application Publication
(10) Pub. No.: US 2011/0275109 A1
(43) Pub. Date: Nov. 10, 2011

(54) Immunity Evaluation Method, Apparatus and Program

(75) Inventors: Katsuiku Hirokawa, Tokyo (JP); Masanori Utsuyama, Tokyo (JP); Masanobu Kitagawa, Tokyo (JP)

(73) Assignee: National University Corporation Tokyo Medical and Dental University, Tokyo (JP)

(21) Appl. No.: 13/140,627

(22) PCT Filed: Dec. 17, 2009
(86) PCT No.: PCT/JP2009/006952

§ 371 Date: Jul. 20, 2011

(30) Foreign Application Priority Data
Dec. 18, 2008 (JP) 2008-321991

(51) Int. Cl. C12Q 1/02; G06F 19/00; C12M 1/34

(52) U.S. Cl. 435/29; 435/287.1; 702/19

(57) ABSTRACT

An immunity evaluation method, apparatus, and program are provided that can evaluate comprehensive immunity with high precision. An immunity evaluation method for evaluating immunity from collected blood includes measuring a number of specific T cells that are CD8 positive and CD28 positive or negative in the collected blood, and determining a T lymphocyte age based on a regression equation on the basis of a correlation between a specific parameter that is dependent on the number of the specific T cells and age, and the number of specific T cells thus measured.

### FIELD OF THE INVENTION

The present invention relates to an immunity evaluation method, apparatus and program for evaluating immunity from collected blood.

### BACKGROUND

Lymphocytes in the blood are cells that play a central role in immunity, and include cells (subsets) having different functions such as T cells, B cells, and natural killer cells (NK cells). In addition, T cells are also not one group, but rather are made up of subsets called CD4 T cells and CD8 T cells which differ functionally from each other.

These cells respectively have characteristic surface proteins (antigens). Therefore, the number and proportion of specific cells have conventionally been measured by dyeing using a monoclonal antibody of these antigens and employing flowcytometry. In addition, functional measurements are performed by measuring a protein (cytokine) related to the proliferative activity or proliferation of each lymphocyte under the cultural conditions. Then, using such a method, the inventors of the present application have found that the configuration of the subsets of lymphocytes and the functions thereof change or decrease with aging (reference is made to the following Patent Document 1 and Non-patent Documents 1 and 2).
Patent Document 1: WO2007/145333 Pamphlet
Non-patent Document 1: M. Utsuyama, K. Hirokawa, C. Kurashima, M. Fukayama, T. Inamatsu, K. Suzuki, W. Hashimoto and K. Sato; “Differential Age-change in the Number of CD4+, CD45RA+ and CD4+CD29+ T Cell Subsets in the Human Peripheral Blood,” Mechanism of Ageing and Development, Vol. 63-1, pp. 57-68, Mar. 15, 1992
Non-patent Document 2: Katsuiku Hirokawa, “Aging and Immunity,” Journal of The Japan Geriatrics Society, vol. 40-6, pp. 543-552, November. 2003

However, although the individual parameters exemplified in these documents express the ratio and function of each subset, they do not necessarily reflect the comprehensive immunity of humans with high precision.

### SUMMARY

The present invention has been made taking the above situation into account, and thus has a first object of providing an immunity evaluation method, apparatus and program that can evaluate comprehensive immunity with high precision. In addition, the present invention has a second object of providing an immunity evaluation method, apparatus and program that can evaluate comprehensive immunity easily and with high precision.

The present invention has involved an unexpected finding that the number of CD28 positive T cells constituting the CD8 positive cells (killer T cells) or the proportion thereof reflects the comprehensive immunity with high precision, thereby arriving at completion of the present invention. This is reflected in several of the summarized invention embodiments as shown and described below.

According to a first aspect of the present invention, an immunity evaluation method for evaluating immunity from collected blood includes:

a measuring step of measuring a number of specific T cells that are CD8 positive and CD28 positive or negative in the collected blood; and

a calculating step of determining T lymphocyte age on the basis of a regression equation based on a correlation between a specific parameter dependent on the number of specific T cells and age, and the number of specific T cells thus measured.

According to the first aspect of the present invention, since a specific parameter that depends on the number of specific T cells that are CD8 positive and CD28 positive or negative is used, the comprehensive immunity can be evaluated with high precision through the T lymphocyte age calculated therefrom.

In addition, the cultivation of lymphocytes is essential to measure the T cell proliferative activity, a result of which a long time of at least three days is required to determine the T cell proliferation index (refer to Patent Document 1), which excels in correlation with the age. However, when measuring the number of specific T cells, since there is little need to perform a process consuming a long time such as cultivation, the comprehensive immunity can be evaluated easily and with high precision, according to the first aspect of the present invention.

It should be noted that “T lymphocyte age” in the present specification is the same as “immunity-adjusted age” disclosed in a prior patent application by the present inventors (PCT/JP2007/062158), and is a marker that determines and evaluates the comprehensive immune function level of humans. However, “T lymphocyte age” differs from “immunity-adjusted age” calculated from the T cell proliferation index in the point of being calculated by measuring the number of specific T cells among the T cells, or the like.

According to a second aspect of the present invention, in the immunity evaluation method as described in the first aspect, the specific T cells are CD8 positive and CD28 positive.

It is known that there is a trend of the number of T cells, the number of CD8 positive T cells and the number of CD28 positive T cells to all decrease with increasing age. According to the second aspect of the present invention, T cells satisfying three conditions having similar trends in this way (T cells that are CD8 positive and CD28 positive) are adopted as the specific T cells; therefore, a regression equation having a higher correlation coefficient is obtained, a result of which the comprehensive immunity can be evaluated with higher precision.

According to a third aspect of the present invention, in the immunity evaluation method as described in the first or second aspect, the specific parameter is at least one selected from a group consisting of the number of the specific T cells per a predetermined amount of blood, and a proportion of the number of specific T cells to a number of CD8 positive cells.

According to the third aspect of the present invention, the comprehensive immunity can be evaluated with higher precision since the number of the specific T cells and/or the proportion of the number of the specific T cells is/are employed as the specific parameter(s).

According to a fourth aspect of the present invention, in the immunity evaluation method as described in any one of the first to third aspects, the calculation step includes a step of determining a predicted value of the number of the specific T cells by substituting into a regression equation an actual age inputted, and determining an estimated range of T lymphocyte ages from the predicted value and the number of specific T cells measured.

According to the fourth aspect of the present invention, the immunity can be easily recognized since the T lymphocyte age is calculated by setting an estimated range having a certain span.

According to a fifth aspect of the present invention, an immunity evaluation method for evaluating immunity includes:

a calculating step of determining an evaluation value based on an immune cell marker corresponding to respective immune cells contained in blood collected; and

an evaluating step of evaluating immunity based on the evaluation value,

in which a specific parameter dependent on a number of specific T cells that are CD8 positive and CD28 positive or negative is used as the immune cell marker in the calculating step.

According to the fifth aspect of the present invention, the comprehensive immunity can be evaluated with high precision through an evaluation value calculated, since a specific parameter that depends on the number of specific T cells that are CD8 positive and CD28 positive or negative is used.

In addition, when measuring the number of specific T cells, since there is little need to perform a process consuming a long time such as cultivation, the comprehensive immunity can be evaluated easily and with high precision.

According to a sixth aspect of the present invention, in the immunity evaluation method as described in the fifth aspect, the specific T cells are CD8 positive and CD28 positive.

According to the sixth aspect of the present invention, the comprehensive immunity can be evaluated with higher precision since CD8 positive and CD28 positive T cells are used as the specific T cells.

According to a seventh aspect of the present invention, in the immunity evaluation method as described in the fifth or sixth aspect, the specific parameter is at least one selected from the group consisting of a number of the specific T cells per a predetermined amount of blood, and a proportion of the number of the specific T cells to a number of CD8 positive cells.

According to the seventh aspect of the present invention, the comprehensive immunity can be evaluated with higher precision since the number of specific T cells and/or the proportion of the number of specific T cells is/are used as the specific parameter(s).

According to an eighth aspect of the present invention, in the immunity evaluation method as described in any one of the fifth to seventh aspects, a marker other than the specific parameter is jointly used as the immune cell marker.

According to the eighth aspect of the present invention, since a marker other than the specific parameter is jointly used, the evaluation value is a value in which immunity is more broadly reflected. As a result, a more comprehensive immunity can be evaluated.

According to a ninth aspect of the present invention, in the immunity evaluation method as described in the eighth aspect, a T cell proliferation index that is dependent on both a number of T cells and a T cell proliferative activity is jointly used as the immune cell marker.

According to the ninth aspect of the present invention, since the T cell proliferation index, which excels in correlation with age, is jointly used, a more comprehensive immunity can be evaluated with high precision.

According to a tenth aspect of the present invention, an immunity evaluation apparatus that evaluates immunity from blood collected includes:

a storage means for storing a regression equation based on correlation between a specific parameter, which is dependent on a number of specific T cells that are CD8 positive and CD28 positive or negative, and age; and

a calculating means for determining a T lymphocyte age based on the regression equation stored in the storage means and the number of specific T cells inputted.

According to an eleventh aspect of the present invention, in the immunity evaluation apparatus as described in the tenth aspect, the specific T cells are CD8 positive and CD28 positive.

According to a twelfth aspect of the present invention, in the immunity evaluation apparatus as described in the tenth or eleventh aspect, the specific parameter is at least one selected from the group consisting of a number of the specific T cells per a predetermined amount of blood, and a proportion of the number of specific T cells to a number of CD8 positive cells.

According to a thirteenth aspect of the present invention, in the immunity evaluation method as described in any one of the tenth to twelfth aspects, the calculating means has an estimated range calculating means for determining a predicted value of the number specific T cells by substituting into the regression equation an actual age inputted, and determining an estimated range of T lymphocyte ages from the predicted value and the number of specific T cells thus measured.

According to a fourteenth aspect of the present invention, an immunity evaluation apparatus for evaluating immunity includes:

a calculating means for determining an evaluation value based on an immune cell marker that corresponds to respective immune cells contained in blood collected; and

an evaluating means for evaluating immunity based on the evaluation value,

in which the calculating means uses a specific parameter that is dependent on a number of specific T cells that are CD8 positive and CD28 positive or negative as the immune cell marker.

According to a fifteenth aspect of the present invention, in the immunity evaluation apparatus as described in the fourteenth aspect, the specific T cells are CD8 positive and CD28 positive.

According to a sixteenth aspect of the present invention, in the immunity evaluation apparatus as described in the fourteenth or fifteenth aspect, the specific parameter is at least one selected from the group consisting of the number of specific T cells per a predetermined amount of blood, and a proportion of the number of specific T cells to a number of CD8 positive cells.

According to a seventeenth aspect of the present invention, in the immunity evaluation apparatus as described in any one of the fourteenth to sixteenth aspects, the calculating means jointly uses a marker other than the specific parameter as the immune cell marker.

According to an eighteenth aspect of the present invention, in the immunity evaluation apparatus as described in the seventeenth aspect, the calculating means jointly uses a T cell proliferation index that depends on both a number of T cells and a T cell proliferative activity as the immune cell marker.

According to a nineteenth aspect of the present invention, in an immunity evaluation program for evaluating immunity from blood collected, the program enables a computer to function as:

a storage means for storing a regression equation based on a correlation between a specific parameter that depends on a number of specific T cells that are CD8 positive and CD28 positive or negative, and age; and

a calculating means for determining a T lymphocyte age based on the regression equation and a number of specific T cells inputted.

According to a twentieth aspect of the present invention, in the immunity evaluation program as described in the nineteenth aspect, the specific T cells are CD8 positive and CD28 positive.

According to a twenty-first aspect of the present invention, in the immunity evaluation program as described in the nineteenth or twentieth aspect, the specific parameter is at least one selected from the group consisting of a number of the specific T cells per a predetermined amount of blood, and a proportion of the number of the specific T cells to a number of CD8 positive cells.

According to a twenty-second aspect of the present invention, in the immunity evaluation program as described in any one of the nineteenth to twenty-first aspects, the calculating means determines a predicted value of the number of the specific T cells by substituting into a regression equation an actual age inputted, and determines an estimated range of T lymphocyte ages based on the predicted value and the number of the specific T cells thus measured.

According to a twenty-third aspect of the present invention, in an immunity evaluation program for evaluating immunity from blood collected, the program enables a computer to function as:

a calculating means for determining an evaluation value based on an immune cell marker that corresponds to respective immune cells contained in the blood collected; and

an evaluation means for evaluating immunity based on the evaluation value,

in which the calculating means is made to use a specific parameter that depends on a number of specific T cells that are CD8 positive and CD28 positive or negative as the immune cell marker.

According to a twenty-fourth aspect of the present invention, in the immunity evaluation program as described in the twenty-third aspect, the specific T cells are CD8 positive and CD28 positive.

According to a twenty-fifth aspect of the present invention, in the immunity evaluation program as described in the twenty-third or twenty-fourth aspect, the specific parameter is at least one selected from the group consisting of a number of the specific T cells per a predetermined amount of blood, and a proportion of the number of the specific T cells to a number of CD8 positive cells.

According to a twenty-sixth aspect of the present invention, in the immunity evaluation program as described in any one of the twenty-third to twenty-fifth aspects, a marker other than the specific parameter is jointly used as the immune cell marker.

According to a twenty-seventh aspect of the present invention, in the immunity evaluation program as described in the twenty-sixth aspect, a T cell proliferation index that depends on both a number of T cells and a T cell proliferative activity is jointly used as the immune cell marker.

According to the present invention, since a specific parameter that depends on the number of specific T cells that are CD8 positive and CD28 positive or negative is used, the comprehensive immunity can be evaluated with high precision through the T lymphocyte age calculated therefrom.

In addition, the cultivation of lymphocytes is essential to measure the T cell proliferative activity, a result of which a long time of at least three days is required to obtain the T cell proliferation index (refer to Patent Document 1), which excels in correlation with the age. However, when measuring the number of specific T cells, since there is little need to perform a process consuming a long time such as cultivation, the comprehensive immunity can be evaluated easily and with high precision according to the present invention.

Further features and advantages of the invention will become clear from the attached claims and the following description, in which the invention is illustrated in detail with reference to the schematic drawings.

### BRIEF DESCRIPTION OF THE DRAWINGS

FIG. 1 is a block diagram of an immunity evaluation apparatus according to one embodiment of the present invention;

FIG. 2 is a block diagram of a main computer unit configuring the immunity evaluation apparatus of FIG. 1;

FIG. 3 is a graph showing a regression equation based on the correlation between a specific parameter and age according to an embodiment of the present invention;

FIG. 4 is a graph showing a regression equation based on the correlation between a specific parameter and T cell proliferative activity according to an embodiment of the present invention;

FIG. 5 is a graph showing a regression equation based on the correlation between a specific parameter and age according to an embodiment of the present invention;

FIG. 6 presents graphs showing regression equations based on the correlation between a specific parameter and the age of healthy persons of respective genders according to an embodiment of the present invention;

FIG. 7 is a graph showing a regression equation based on the correlation between a specific parameter and T cell proliferation index according to an embodiment of the present invention;

FIG. 8 presents graphs showing regression equations based on the correlation between a parameter and T cell proliferative activity according to a comparative example;

FIG. 9 is a graph showing a regression equation based on the correlation between a specific parameter and age according to an embodiment of the present invention;

FIG. 10 is a graph showing a regression equation based on the correlation between a specific parameter and age according to an embodiment of the present invention;

FIG. 11 is a flowchart showing an evaluation process for immunity performed by the main computer unit of FIG. 2;

FIG. 12 is an evaluation table of immunity evaluation ranking and T lymphocyte ages relative to measured values and predicted values of the specific parameter according to an embodiment of the present invention; and

FIG. 13 is a graph showing an example of range demarcating for age indication.

### DETAILED DESCRIPTION

For a more complete understanding and appreciation of the invention and many of its advantages, reference will be made to the following Detailed Description taken in conjunction with the accompanying drawings.

### Hardware Configuration

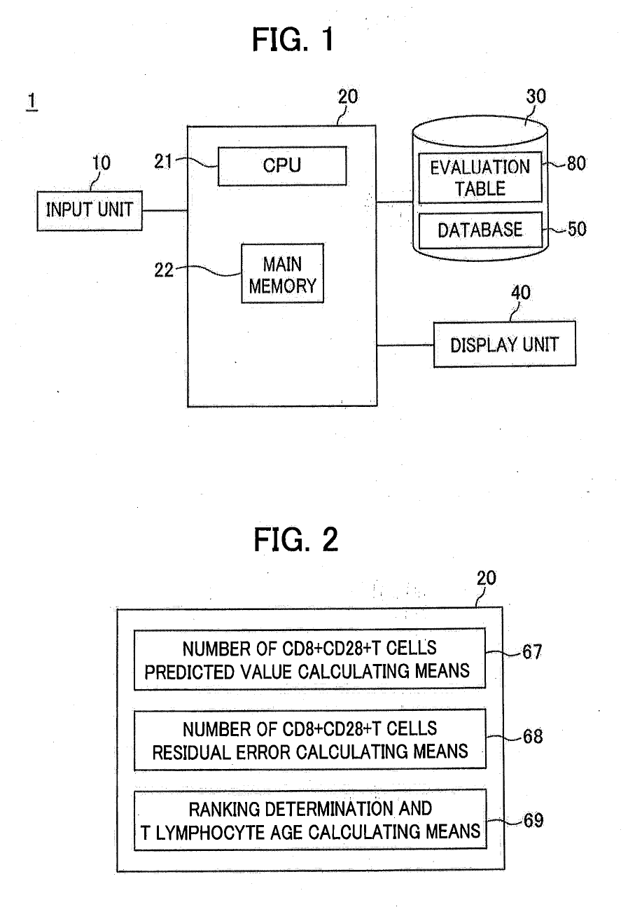

FIG. 1 is a block diagram of an immunity evaluation apparatus 1 according to a contemplated embodiment of the present invention, and FIG. 2 is a block diagram of a main computer unit 20.

As shown in FIG. 1, the immunity evaluation apparatus 1 includes an input unit 10, the main computer unit 20, an external storage device 30 as a storage unit, and a display unit 40. The input unit 10 includes a keyboard, mouse, and the like that allow for relevant information of persons subjected to evaluation of immunity (persons whose blood has been sampled), a specific parameter described later or immunity cell marker value, relevant information of healthy persons, and the like to be inputted. The name, address, gender, actual age, past illnesses, present illnesses, etc. of a person subjected to evaluation of immunity can be exemplified as relevant information of a person subject to evaluation of immunity.

In addition to a CPU (central processing unit) 21, the main computer unit 20 includes main memory 22 and an interface circuit (not shown) for connecting with external circuits.

The external storage device 30 is a hard disk drive, for example. A database 50, evaluation table 80, and the like are stored along with an immunity evaluation program described later in this external storage device 30, and the contents thereof are read from the main memory 22 of the main computer unit 20 and executed as necessary.

The database 50 stores data associating the specific parameter or immunity cell marker values of healthy persons serving as specific reference values corresponding to a plurality of immune cells with relevant information of the healthy persons, and is a structure that allows for the specific parameter or immunity cell marker values of a new healthy person to be successively added and stored using the input unit 10. Herein, healthy person indicates a person for which no special anomalies have been recognized in a general medical examination, and the relevant information of a healthy person indicates the name, address, gender, actual age, body parameters such as weight and height, specific parameter or immune cell marker values, and the like.

### Function Configuration

The main computer unit 20 is made to function as a storage means for storing regression equations based on the correlation between a specific parameter and age, and as a calculating means for determining T lymphocyte ages based on the regression equation and the number of specific T cells inputted, according to the immunity evaluation process stored in the above hardware configuration.

More specifically, the program according to the present embodiment causes the main computer unit 20 to function as a specific parameter predicted value calculating means 67, a specific parameter residual error calculating means 68, and a rank determining and T lymphocyte age calculating means 69 based on the specific parameter, as shown in FIG. 2. The details of each function are further described below

### Immunity Evaluation Method

### First Mode

The immunity evaluation method according to the present mode includes a measurement step of measuring a number of specific T cells in collected blood, and a calculating step of obtaining a T lymphocyte age on the basis of a regression equation based on the correlation between the specific parameter and age, and the number of specific T cells thus measured. Using the specific parameter dependent on the number of specific T cells, which are CD8 positive and CD28 positive or negative, it is possible to evaluate the comprehensive immunity with high precision through the T lymphocyte age calculated.

The measurement step may be achieved by performing flowcytometric analysis on peripheral blood collected.

### Flowcytometric Analysis

Flowcytometric analysis is performed based on the peripheral blood collected.

1. Fifty (μL) of peripheral blood is dispensed into a test tube.

2. A fluorescent-labeled antibody solution is added to the test tube.

3. The test tube is left in a dark location for 30 minutes.

4. Two (ml) of a hemolysis agent is added and agitated, then left for 10 minutes to allow for the red blood cells to dissolve.

5. Three (ml) of a PBS solution is added and centrifuged at 1200 (rpm) for 5 minutes.

6. The supernatent is removed by suction.

7. Five-hundred (μL) of PBS solution is added to prepare the cell suspension by causing the cells to resuspend.

8. The fluorescent-labeled antibody positive cell index is calculated by flowcytometry using equipment dedicated software.

The above-mentioned fluorescent-labeled antibodies used in flowcytometry may be appropriately selected, and may be PE-CD3/FITC-CD8/PC5-CD28, for example. In this case, the cells for which fluorescence of any of FITC, PE and PC5 is detected can be determined to be CD8 positive and CD28 positive T cells, and the cells for which the fluorescence of FITC and PE is detected but the fluorescent of PC5 is not detected can be determined to be CD8 positive and CD28 negative T cells.

Herein, CD8 positive and CD28 positive T cells and CD8 positive and CD28 negative T cells are exemplified as the specific T cells. It is known that there is a trend of the number of T cells, the number of CD8 positive T cells and the number of CD28 positive T cells all decreasing with increasing age. According to the second aspect of the invention, T cells satisfying three conditions having similar trends in this way (T cells that are CD8 positive and CD28 positive) are adopted as the specific T cells; therefore, a regression equation having a higher correlation coefficient is obtained, a result of which the comprehensive immunity can be evaluated with higher precision. Therefore, CD8 positive and CD28 positive T cells are preferable in the point of allowing for the comprehensive immunity to be evaluated with higher precision.

In the calculating step, the T lymphocyte age is determined on the basis of the regression equation based on the correlation between the specific parameter dependent on the number of the specific T cells and the age, and the number of the specific T cells measured. Herein, the specific parameter is not particularly limited so long as being a variable that depends on the number of the specific T cells, and may or may not be the number of the specific T cells itself. However, the specific parameter is preferably at least one selected from the group consisting of a number of the specific T cells per predetermined amount of blood, and a ratio of the specific T cells to a number of CD8 positive cells, from the viewpoint of allowing for the comprehensive immunity to be evaluated with higher precision.

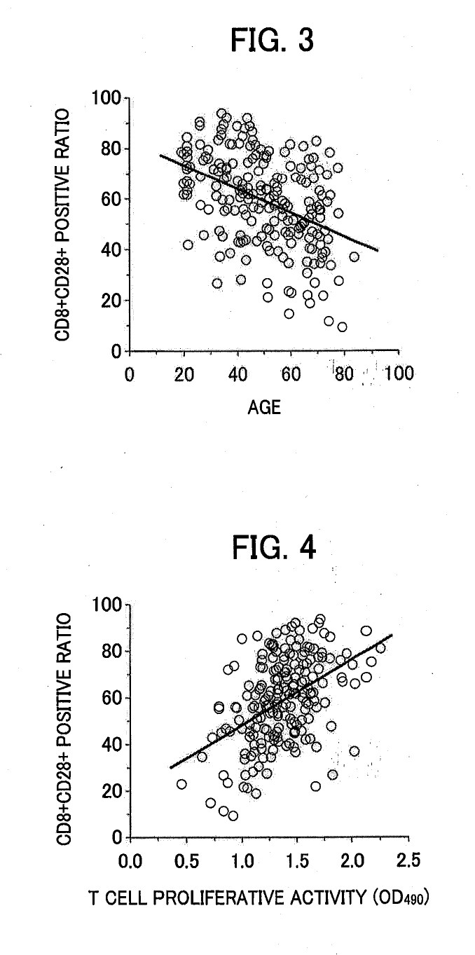

A regression equation based on the correlation between the proportion of the number of specific T cells to the number of CD8 positive cells and age was prepared based on the data obtained from approximately 300 healthy persons, as shown in FIG. 3. It should be noted that the specific T cells in FIG. 3 are T cells of CD8 positive and CD28 positive, and the above-mentioned proportion is called the CD8⁺CD28⁺ positive ratio. The regression equation shown in FIG. 3 is y=−0.47x+82.8 (where x is the age, and y is the predicted value of the CD8⁺CD28⁺ positive ratio), and has a high correlation coefficient (R=0.43).

In addition, a regression equation based on the correlation between the CD8⁺CD28⁺ positive ratio and the T cell proliferative activity was prepared based on the data obtained from approximately 300 healthy persons, as shown in FIG. 4. It should be noted that the T cell proliferative activity was calculated according to the following procedure.

In a case of calculating the T cell proliferation index, the following procedure may be performed.

### Collection of Mononuclear Cells

1. Eight milliliters of blood is collected into a CPT mononuclear cell preparation vacuum tube (Becton, Dickinson and Company: 8362761).

2. The sample is centrifuged at 3000 (rpm) for 20 Minutes at room temperature.

3. A lymphocyte layer obtained from centrifuging is collected.

4. Physiological saline is added to the lymphocyte layer thus collected.

5. Centrifuging is performed at 1500 (rpm) for 10 minutes at room temperature.

6. The supernatant is discarded and physiological saline is added thereto again.

7. Centrifuging is performed at 1200 (rpm) for 5 minutes at room temperature.

8. The supernatant is discarded, and the cell culture fluid (RPMI-1640) is added to make a cell suspension.

9. The cell concentration is measured (using a 0.2% Trypanblue solution).

10. The cell concentration in the cell suspension is adjusted to 1×10⁶/mL.

### Anti-CD3 Antibody Stimulation

Anti-CD3 antibody is coated on 96-well plates.

1. Thirty microliters of Orthoclone OKT3 solution (ORTHO BIOTECH: 672993402) is diluted at a proportion with 10 (mL) of physiological saline.

2. The diluted OKT3 solution is placed in the 96-well plate at 100 (μl/well).

3. The solution is left to stand for 2 hours at room temperature.

4. The diluted OKT3 solution is discarded by suction, and physiological saline is placed in the 96-well plate at 200 (μL/well).

5. The physiological saline is discarded by suction, and physiological saline is placed in the 96-well plate at 200 (μL/well) again.

6. This operation is repeated 5 times in total.

7. Physiological saline is placed in the 96-well plate at 200 (μL/well), and kept in cold storage until used.

### T Cell Proliferative Activity Analysis by MTS Method

1. One-hundred microliters per well of 10% FBS-RPMI and 100 (μL/well) of a 1×10⁶/mL cell suspension are respectively placed in the 96-well plate prepared by coating the above-mentioned CD3 antibody. Three wells are used for each sample.

2. The samples are cultured in a cell incubator at conditions of 5% carbon dioxide gas at 37° C.

3. Forty microliters per well of an MTS solution is added at the time of 68-hours culturing.

4. At the time of 72-hours culturing, measurement is performed by colorimetry (490 nm).

The above-mentioned T cell proliferative activity is thereby calculated.

The regression equation shown in FIG. 4 is y=28.3x+20.0 (where x is the T cell proliferative activity, and y is the predicted value of the CD8⁺CD28⁺ positive ratio), and has a high correlation coefficient (R=0.46). Since the T cell proliferative activity is a kind of parameter excelling in correlation with the age (refer to Patent Document 1), it is reconfirmed that the CD8⁺CD28⁺ positive ratio reflects the immunity with high precision. In addition, although cell cultivation over the above-mentioned such 72 hours is required in the measurement of the T cell proliferative activity and the T cell proliferation index, such cell cultivation is not necessary in the measurement of the number of specific T cells, which can be completed easily.

Next, each age is input to the regression equation shown in FIG. 3, the predicted value of the CD8⁺CD28⁺ positive ratio at each age is determined, the difference between the predicted value thus obtained and the measured value is determined, and the residual error is determined. In other words, the residual error is determined based on the expression of measured value (CD8⁺CD28⁺ positive ratio)−predicted value (value obtained from regression equation).

Next, the T lymphocyte age is calculated. The T lymphocyte age according to the computational expression (calculated age) is determined by substituting the measured value for the CD8⁺CD28⁺ positive ratio of subjects into the following expression in which the previously described regression equation has been converted.

Calculated age=(measured value−82.8)/0.47

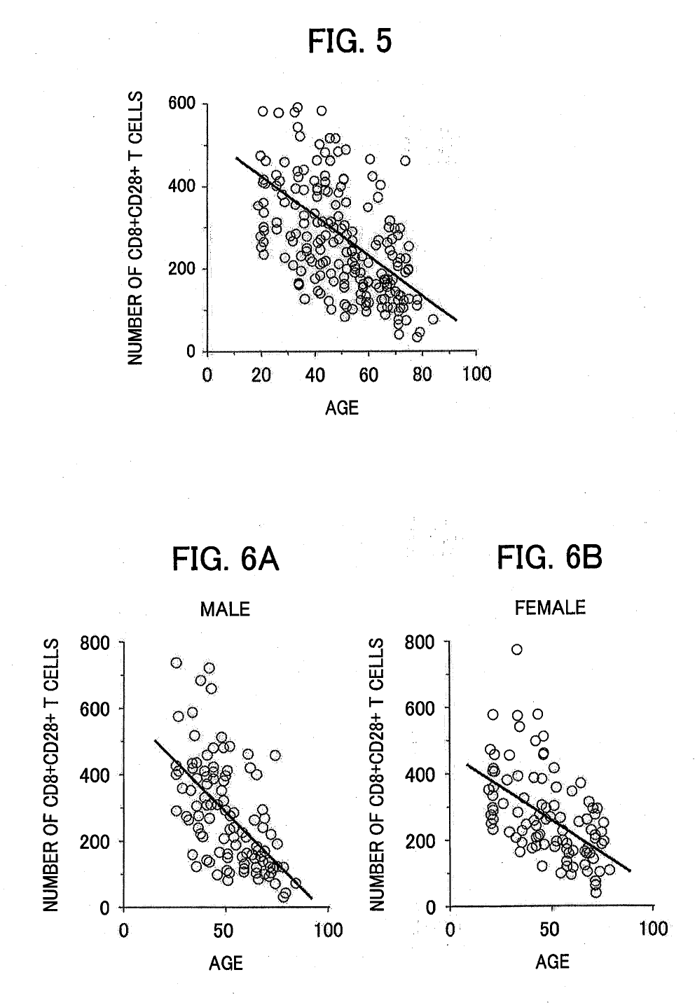

Similarly, a regression equation based on the correlation between the number of CD8 positive and CD28 positive T cells per a predetermined amount of blood and the age was prepared based on the data obtained from approximately 300 healthy persons, and is shown in FIG. 5. It should be noted that the predetermined amount is 1 μL in FIG. 5; however, it is not limited to this and may be any amount. The regression equation shown in FIG. 5 is y=−4.87x+523 (where x is the age, and y is the predicted value of for the number of CD8 positive and CD28 positive T cells per 1 μL of blood), and has a high correlation coefficient (R=0.50).

Herein, the results of classifying the data obtained from approximately 300 healthy persons according to the gender of the subject are shown in FIG. 6. The regression equation between male age and number of CD8 positive and CD28 positive T cells per a predetermined amount of blood shown in FIG. 6(a) was y=−6.089x+597.0 (where x is the male age, and y is the predicted value for the number of CD8 positive and CD28 positive T cells per 1 μL of blood; and R is 0.543); whereas, the regression equation between female age and the number of specific T cells per a predetermined amount of blood shown in FIG. 6(b) was y=−4.136x+476.9 (where x is the female age, and y is the predetermined value for the number of CD8 positive and CD28 positive T cells per 1 μL of blood; and R is 0.477). These results indicate that the decline in immunity accompanying increasing age is more gradual for females than males, and further, the fact that females have a longer life-span than males is accurately reflected.

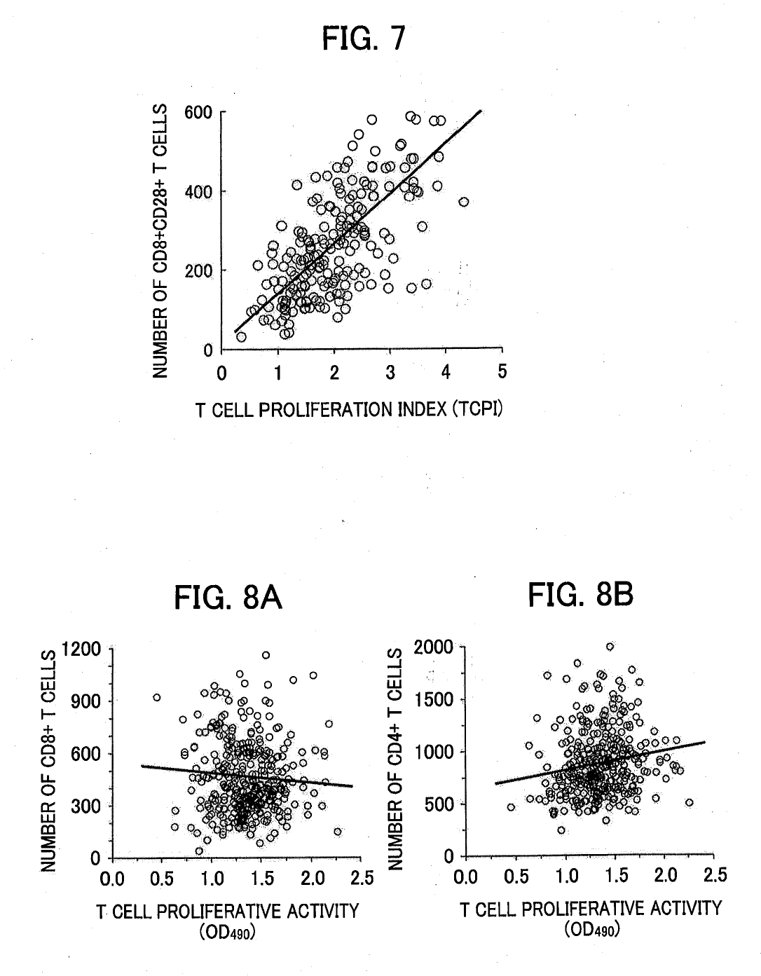

In addition, the regression equation between the T cell proliferation index (TCPI) and the number of CD8 positive and CD28 positive T cells per 1 μL of blood for each of the approximately 300 healthy persons was determined, and these results are shown in FIG. 7. It should be noted that it is known that the T cell proliferation index is a parameter excelling in correlation with age and reflects immunity with high precision (refer to Patent Document 1). More specifically, the T cell proliferation index multiplies the number of T cells with the T cell proliferative activity, and can be determined from the following equation, for example. It should be noted that the OD value in the equation is a value obtained by subtracting the OD value of proliferated cells without stimulation from the OD value obtained in cells proliferated by stimulation.

T cell proliferation index=OD(490 nm)×(number of T cells in peripheral blood(per μL/1000

The regression equation shown in FIG. 7 is y=125.7x+16.4 (where x is the T cell proliferation index, and y is the predicted value for the number of CD8 positive and CD28 positive T cells per 1 μL of blood), and has a high correlation coefficient (R=0.69). The predominance of the specific parameter of the number of CD8 positive and CD28 positive T cells per predetermined amount of blood is evident from this as well.

In contrast, the regression equation between the number of CD8 positive T cells or the number of CD4 positive T cells per a predetermined amount of blood and the T cell proliferative activity is as shown in FIG. 8. The regression equation shown in FIG. 8(a) is y=−55.9x+546 (where x is the T cell proliferative activity, and y is the predicted value for the number of CD8 positive T cells per 1 μL of blood), and the correlation coefficient is extremely low (R=0.06). In addition, the regression equation shown in FIG. 8(b) is y=179.8x+639 (where x is the T cell proliferative activity, and y is the predicted value for the number of CD4 positive T cells per 1 μL of blood), and the correlation coefficient is low (R=0.15). It thereby became evident that the extent to which immunity is reflected by the parameter of the number of CD8 positive T cells or the number of CD4 positive T cells per predetermined amount of blood is a low.

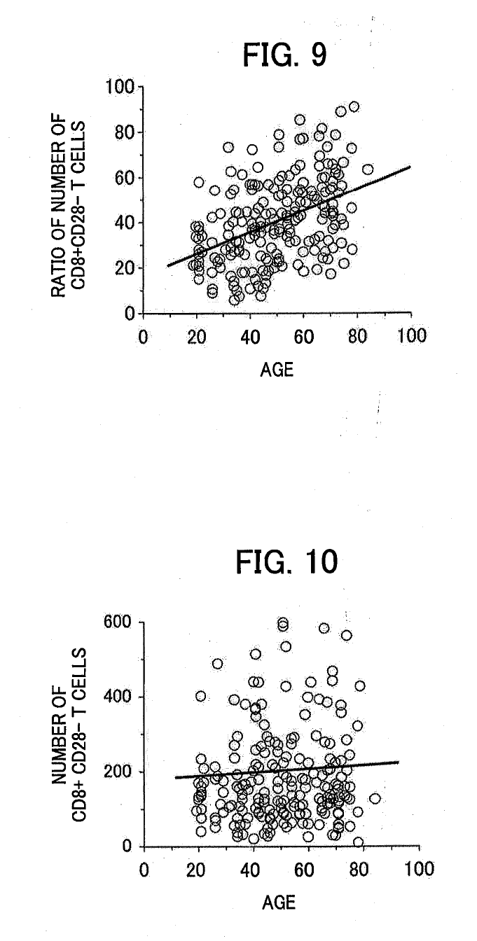

In addition, regression equations between the percentage of the number of CD28 negative T cells among the number of CD8 positive T cells, or the number of CD8 positive-CD28 negative T cells per a predetermined amount of blood, and the T cell proliferative activity are as shown in FIGS. 9 and 10. The regression equation shown in FIG. 9 has a high correlation coefficient (R=0.43). The regression equation shown in FIG. 10 has a low correlation coefficient (R=0.05). Although the specific parameter of the number of CD8 positive and CD28 negative T cells per predetermined amount of blood is superior to non-specific parameters, it is inferior to the number of CD8 positive and CD28 positive T cells per predetermined amount of blood.

Next, each age is input to the regression equation shown in FIG. 5, the predicted value for the specific T cells per 1 μL of blood is determined for each age, the difference between the predicted values thus obtained and the measured value is determined, and the residual error is determined. In other words, the residual error is determined based on the expression of measured value (specific T cells per 1 μL of blood)−predicted value (value obtained from regression equation).

Next, the T lymphocyte age is calculated. The T lymphocyte age according to the computational expression (calculated age) is determined by substituting the measured value for the specific T cells per 1 μL of blood of a subject into the following expression in which the previously described regression equation has been converted.

Calculated age=(523−measured value)/4.87)

However, the calculated age obtained in this way is an estimated value, and it would be more realistic to express with a certain degree of range. Therefore, as shown next, the range of T lymphocyte ages are established by the residual error, which is the difference between the measured value for the CD8⁺CD28⁺ positive ratio or specific T cells per 1 μL of blood and the predicted values thereof, being expressed as a normal distribution.

Since the difference between the predicted value obtained from the actual age of subjects and the measured value, i.e. residual error, is expressed as a normal distribution, the standard deviation of the residual error can be determined using the below formula.

$${σ}^{′ 2} = \frac{1}{n - 1} {\sum }_{i = 1}^{n} {( {χ}_{1} - \overline{χ} )}^{2}$$

Based on the standard deviation SB of the residual error thus obtained, the ranges of age indication are divided into ±0.5 SD, ±1.0 SD, ±1.5 SD, and ±2.0 SD, as shown in FIG. 13. In this case, the residual error is ±45 (0.5 SD), ±90 (1.0 SD), ±135 (1.5 SD), and ±180 (2.0 SD).

The gap between the actual age and T lymphocyte age of subjects is calculated from the residual errors with these standard deviations as a basis. First, the calculated age is determined using the aforementioned equation, and when observing the difference from the actual ages, the values determined from the standard deviation at any age are substantially the same, and thus for each age, ±0.5 SD is approximately ±15 years, ±1.0 SD is approximately ±30 years, ±1.5 SD is approximately ±45 years, ±2.0 SD is approximately ±60, and ±2.0 SD or more is approximately ±60 years or more.

Since it is set so that the T lymphocyte age corresponds to the value of the actual age, and so that evaluation can be performed from approximately 17 to 99 years of age, the difference between the calculated age obtained as described above and the actual age is assumed to be 20% and is referred to as indicated age. Specifically, the indicated age is obtained using the following equation.

Indicated age=actual age+(calculated age−actual age)×0.2

The immunity evaluation apparatus 1 executes the following steps according to the above-mentioned immunity evaluation program.

(1) A step of calculating a predicted value of a specific parameter by substituting into the regression equation obtained and stored in advance the actual age inputted (specific parameter predicted value calculating means 67).

(2) A step of calculating the residual error of the specific parameter based on the measured value of the specific parameter inputted and the predicted value thereof (specific parameter residual error calculating means 68).

(3) A step of rank determining and calculating the T lymphocyte age based on the residual error obtained (rank determining and T lymphocyte age calculating means 69).

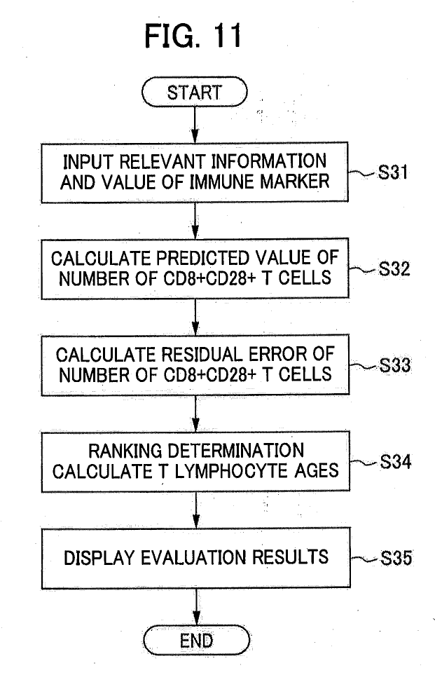

FIG. 11 is a flowchart illustrating an example of the evaluation process for immunity performed by the main computer unit 20 based on the measured value of a specific parameter and the actual age inputted at the input unit 10.

First, a specific parameter value along with relevant information such as the address, name, actual age, gender, past illnesses and present illnesses of a person subjected to evaluation of immunity are inputted using the input unit 10, and then the enter key is pressed (Step S31). Then, a predicted value of the specific parameter is calculated by substituting the actual age inputted to the regression equation obtained and stored in advance (Step S32).

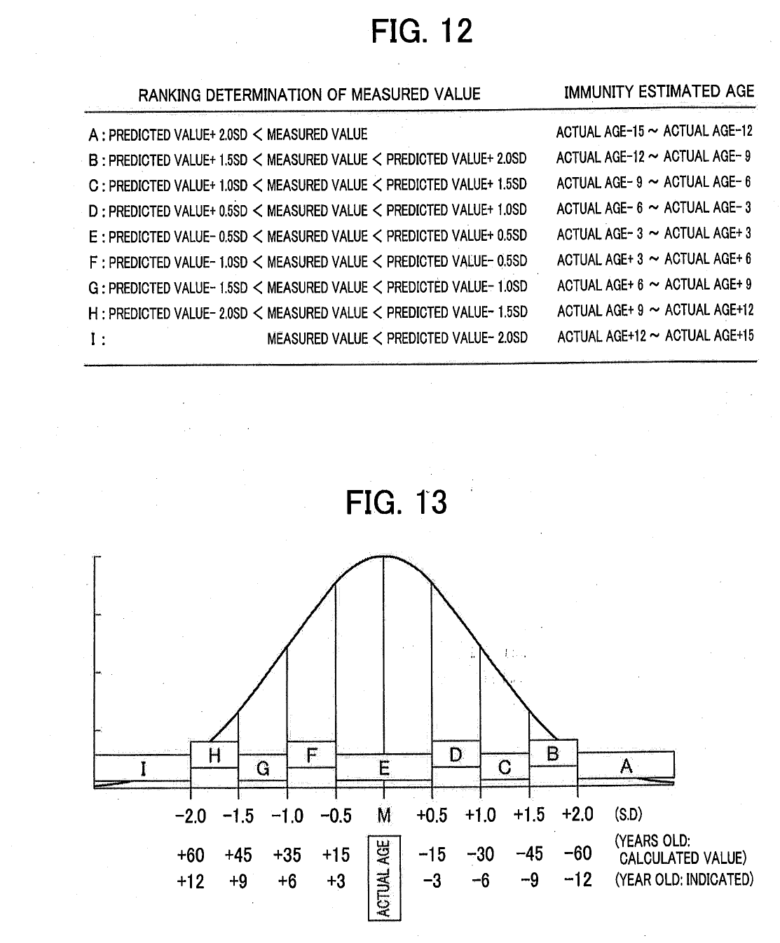

The residual error is calculated based on the predicted value obtained in Step S32 and the measured value of the specific parameter inputted (Step S33). Rankings A to I of the measured value such as that shown in FIG. 12 is determined from the residual error thus obtained, i.e. predicted value and measured value, and the T lymphocyte age is determined (Step S34). Specifically, an evaluation table of the immunity evaluation rankings relative to the measured values and predicted values of the specific parameter and T lymphocyte ages, such as that shown in FIG. 12, is stored in the evaluation table 80. In Step S34, ranking determination is performed based on the measured values and predicted values from this evaluation table, and the T lymphocyte age is calculated. It should be noted that, among the T lymphocyte ages in FIG. 12, the youngest age range is set as ages 17 to 20 and the oldest age range is set as ages 96 to 99.

The above evaluation results are displayed on the display unit 40 (Step S35), and the processing ends. The evaluation contents can be exemplified by “relevant information including the actual age of subjects”, “measured value of the specific parameter”, “predicted value of the specific parameter”, “residual error”, “immunity evaluation ranking”, “T lymphocyte age”, and the like. It should be noted that Patent Document 1 may be referred to for the details of the steps themselves.

### Second Mode

The immunity evaluation method according to the present mode includes a calculating step of determining evaluation values based on an immune cell marker corresponding to respective immune cells contained in the collected blood, and an evaluation step of evaluating immunity based on the evaluation values. In the calculating step, a specific parameter that depends on the number of specific T cells, which are CD8 positive and CD28 positive or negative, is used as the immune cell marker. By using a specific parameter that is dependent on the number of specific T cells that are CD8 positive and CD28 positive or negative, the comprehensive immunity can be evaluated with high precision through the evaluation values thus calculated.

Similarly to the first mode, it is preferable to use CD8 positive and CD28 positive T cells as the specific T cells, and the specific parameter is preferably at least one selected from the group consisting of the number of the specific T cells per a predetermined amount of blood, and a proportion of the number of the specific T cells to a number of CD8 positive cells.

Herein, “immune cell marker” corresponds to a plurality of immune cells contained in the collected blood. The immune cell marker is not particularly limited so long as containing the specific parameter. From the viewpoint of excelling in correlation with age, it is preferable to jointly use at least one selected from the group consisting of the number of T cells per unit blood amount, T cell proliferation index, CD4 T cell/CD8 T cell ratio, number of naïve T cells per unit blood amount, naïve T cell/memory T cell ratio, number of B cells per unit blood amount, and number of NK (natural killer) cells per unit blood amount, and it is particularly preferable to jointly use with the T cell proliferation index. It should be noted that Patent Document 1, incorporated herein by reference, may be referred to for the measurement step of each marker.

In a case of jointly using a marker other than the specific parameter as the immune cell marker, combination with such an immune cell marker may be selected as a combination corresponding to an illness of the subject (marker having a strong tendency of changing greatly with contracting the illness thereof), and may be selected as a combination corresponding to the actual age (marker having a strong tendency of greatly changing in response to an increase in age).

The evaluation value based on each immune cell marker may be the measurement itself, and the measurement may be scored. In one example of scoring the measured values, the values of the immune cell markers of the healthy persons are classified into three levels consisting of a range of cumulative frequencies less than 10%, a range of cumulative frequencies of at least 10% to less than 40%, and a range of cumulative frequencies of at least 40%, and progressively fewer points are assigned from the high classification to low classification of immunity. By classifying into three levels based on such cumulative frequencies, it is possible to accurately carry out scoring.

More specifically, with the value at the cumulative frequency of 10% and the value at the cumulative frequency of 40% as references, 1 point is assigned to a value with the cumulative frequency no more than 10%, 2 points are assigned to a value with the cumulative frequency between 10% and 40%, and 3 points are assigned to a value with the cumulative frequency exceeding 40%. In other words, for the values of healthy persons, the range of cumulative frequencies less than 10% is set to 1 point, indicating a low immunity level, the range of cumulative frequencies of at least 10% and less than 40% is set to 2 points, indicating a medium immunity level, and the range of cumulative frequencies of at least 40% is set to 3 points, indicating a sufficiently high immunity level.

In the present mode, although 1, 2 and 3 points are assigned to the three classifications, respectively, it is not limited thereto, and a high score corresponding to a high immunity level and a low score corresponding to a low immunity level may be assigned. It should be noted that the above-mentioned cumulative frequency of 10% and cumulative frequency of 40% may change somewhat accompanying an increase in the number of healthy persons stored in the database 50.

The evaluation value obtained in this way may be comparatively examined individually by displaying as a radar graph or the like, and may be combined in a sequence such as that illustrated in Patent Document 1.

The features of the invention disclosed in the above description, in the drawings, and in the claims, can be essential to implementing the invention in its various embodiments both individually and in any combination. Those skilled in the art will realize that this invention is capable of embodiments that are different from those shown and that details of the disclosed methods, structures, and descriptions can be changed in various manners without departing from the scope of the invention. Accordingly, the drawings and descriptions are to be regarded as including such equivalent configurations, methods, and arrangements as do not depart from the spirit and scope of the invention.

1. An immunity evaluation method for evaluating immunity from collected blood, the method comprising:
measuring a number of specific T cells that are CD8 positive and CD28 positive in the collected blood;
determining T lymphocyte age on the basis of a regression equation based on a correlation between a specific parameter dependent on the number of specific T cells and age, and the number of specific T cells thus measured; and
the specific parameter is at least one selected from the group consisting of a number of the specific T cells per a predetermined amount of blood and a proportion of the number of specific T cells to a number of CD8 positive cells.

2. The immunity evaluation method of claim 1 further comprising:
determining a predicted value of the number of the specific T cells by substituting into a regression equation an actual age inputted; and
determining an estimated range of T lymphocyte ages from the predicted value and the number of specific T cells measured.

3. An immunity evaluation method for evaluating immunity comprising:
determining an evaluation value based on an immune cell marker corresponding to respective immune cells contained in blood collected;
evaluating immunity based on the evaluation value;
establishing as the immune cell marker a specific parameter dependent on a number of specific T cells that are CD8 positive and CD28 positive; and
the specific parameter being at least one selected from the group consisting of a number of the specific T cells per a predetermined amount of blood and a proportion of the number of the specific T cells to a number of CD8 positive cells.

4. The immunity evaluation method of claim 3, a marker other than the specific parameter is jointly used as the immune cell marker.

5. The immunity evaluation method of claim 4, a T cell proliferation index that is dependent on both a number of T cells and a T cell proliferative activity being jointly used as the immune cell marker.

6. An immunity evaluation apparatus for evaluating immunity from blood collected, said apparatus comprising:
a storage means for storing a regression equation based on a correlation between a specific parameter, said specific parameter being dependent on a number of specific T cells in a sample that are CD8 positive and CD28 positive, and age;
a calculating means for determining a T lymphocyte age based on the regression equation stored in said storage means and the number of specific T cells inputted; and
the specific parameter being at least one selected from the group consisting of a number of the specific T cells per a predetermined amount of blood and a proportion of the number of specific T cells to a number of CD8 positive cells.

7. The immunity evaluation apparatus of claim 6, said calculating means having an estimated range calculating means for determining a predicted value of the number of specific T cells by substituting into the regression equation an actual age inputted, and determining an estimated range of T lymphocyte ages from the predicted value and the number of specific T cells thus measured.

8. An immunity evaluation apparatus for evaluating immunity, said apparatus comprising:
a calculating means for determining an evaluation value based on an immune cell marker that corresponds to respective immune cells contained in blood collected;
an evaluating means for evaluating immunity based on the evaluation value;
said calculating means using a specific parameter that is dependent on a number of specific T cells that are CD8 positive and CD28 positive as the immune cell marker; and
the specific parameter being at least one selected from the group consisting of the number of specific T cells per a predetermined amount of blood and a proportion of the number of specific T cells to a number of CD8 positive cells.

9. The immunity evaluation apparatus of claim 8, said calculating means jointly using a marker other than the specific parameter as the immune cell marker.

10. The immunity evaluation apparatus of claim 9, said calculating means jointly using a T cell proliferation index that depends on both a number of T cells and a T cell proliferative activity as the immune cell marker.

11. An immunity evaluation program for evaluating immunity from blood collected comprising:
said program enabling a computer to function as a storage means for storing a regression equation based on a correlation between a specific parameter that depends on a number of specific T cells that are CD8 positive and CD28 positive, and age;
said program enabling said computer to operate as a calculating means for determining a T lymphocyte age based on the regression equation and a number of specific T cells inputted; and
the specific parameter being at least one selected from the group consisting of a number of the specific T cells per a predetermined amount of blood and a proportion of the number of the specific T cells to a number of CD8 positive cells.

12. The immunity evaluation program of claim 11 further comprising:
said calculating means determines a predicted value of the number of the specific T cells by substituting into a regression equation an actual age inputted; and
said calculating means determines an estimated range of T lymphocyte ages based on the predicted value and the number of the specific T cells thus measured.

13. An immunity evaluation program for evaluating immunity from blood collected comprising:
said program enables a computer to function as a calculating means for determining an evaluation value based on an immune cell marker that corresponds to respective immune cells contained in the blood collected;
said program enables the computer to function as an evaluation means for evaluating immunity based on the evaluation value;
said calculating means is made to use a specific parameter that depends on a number of specific T cells that are CD8 positive and CD28 positive as the immune cell marker; and
the specific parameter is at least one selected from the group consisting of a number of the specific T cells per a predetermined amount of blood and a proportion of the number of the specific T cells to a number of CD8 positive cells.

14. The immunity evaluation program of claim 13, a marker other than the specific parameter being jointly used as the immune cell marker.

15. The immunity evaluation program of claim 14, a T cell proliferation index that depends on both a number of T cells and a T cell proliferative activity being jointly used as the immune cell marker.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)
